(19) United States
(12) Patent Application Publication
(10) Pub. No.: US 2010/0048942 A1
(43) Pub. Date: Feb. 25, 2010

(54) PROCESS FOR PREPARING ISOCYANATES

(75) Inventors: Carsten Knoesche, Niederkirchen (DE); Torsten Mattke, Freinsheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/515,554

(22) PCT Filed: Nov. 30, 2007
(86) PCT No.: PCT/EP07/63070

§ 371 Date: May 20, 2009

(30) Foreign Application Priority Data
Dec. 11, 2006 (EP) 06125811.7

(51) Int. Cl. C07C 263/10; B01J 19/00; B01F 3/04

(52) U.S. Cl. 560/347; 422/129; 366/342; 261/108

(57) ABSTRACT

The present invention relates to a process for preparing isocyanates in particular apparatuses.

The present invention relates to a process for preparing isocyanates in particular apparatuses.

The preparation of isocyanates by phosgenation of the corresponding amines has been known for a long time. A distinction is typically made between phosgenations in the gas phase and in the liquid phase.

In all these processes, solids are formed during the reaction. These can lead to deposits through to blockages. Among other things, sparingly soluble hydrochlorides are formed in phosgenation in the liquid phase from the hydrogen chloride liberated in the reaction and the amine used. Furthermore, the intermediates formed in the reaction, e.g. carbamoyl chlorides, can be solids under the reaction conditions in these processes.

In all phosgenations, solid ureas can be formed by contact of intermediates and also the isocyanates with the amine used. Subsequent products of the isocyanate, e.g. diimides and isocyanurates, are also frequently solid under the reaction conditions.

To eliminate such precipitates, GB 1165831 proposes a reaction in a reactor having a stirring device for, for example, the reaction of aromatic or aliphatic amines in the gas phase.

This has the disadvantage that mechanical stirring devices are particularly susceptible to malfunctions, in particular under the conditions of gas-phase phosgenation.

EP 289840 B1 describes carrying out a gas-phase phosgenation in a reaction space without moving parts. The deposition of blockage-producing particles is prevented here by means of turbulent flow.

EP 928785 B1 reduces solids formation by use of complicated microstructured mixers.

It was an object of the present invention to provide a process by means of which the formation of deposits in an apparatus for preparing isocyanates by phosgenation of amines is prevented.

This object is achieved by a process for preparing diisocyanates by reaction of the corresponding amines with phosgene, if appropriate in the presence of inerts, in which the reaction takes place at least partly in an apparatus whose fluid contact surface has an average peak-to-valley height Rz in accordance with DIN EN ISO 4287 (October 1998 version) of not more than 10 μm.

Particles which may be formed do not adhere or adhere only slightly to surfaces having a low roughness according to the invention, so that the tendency to form deposits is reduced or even nonexistent.

The amines can be monoamines, diamines, triamines or higher-functionality amines, preferably diamines. The corresponding monoisocyanates, diisocyanates, triisocyanates or higher-functionality isocyanates, preferably diisocyanates, are accordingly formed.

The amines and isocyanates can be aliphatic, cycloaliphatic or aromatic, preferably aliphatic or cycloaliphatic and particularly preferably aliphatic.

Cycloaliphatic isocyanates are ones which comprise at least one cycloaliphatic ring system.

Aliphatic isocyanates are ones which have exclusively isocyanate groups bound to straight or branched chains.

Aromatic isocyanates are ones which have at least one isocyanate group bound to at least one aromatic ring system.

The expression (cyclo)aliphatic isocyanates is, for the purposes of the present patent application, short for cycloaliphatic and/or aliphatic isocyanates.

Examples of aromatic diisocyanates are preferably ones having 6-20 carbon atoms, for example monomeric 2,4′- or 4,4′-methylenedi(phenyl isocyanate) (MDI), tolylene 2,4- and/or 2,6-diisocyanate (TDI), o-, m- or p-xylylene diisocyanate (XDI) and 1,5- or 1,8-naphthylene diisocyanate (NDI).

Diisocyanates are preferably (cyclo)aliphatic diisocyanates, particularly preferably (cyclo)aliphatic diisocyanates having from 4 to 20 carbon atoms.

Examples of customary diisocyanates are aliphatic diisocyanates such as tetramethylene 1,4-diisocyanate, pentamethylene 1,5-diisocyanate, hexamethylene 1,6-diisocyanate (1,6-diisocyanatohexane), octamethylene 1,8-diisocyanate, decamethylene 1,10-diisocyanate, dodecamethylene 1,12-diisocyanate, tetradecamethylene 1,14-diisocyanate, derivatives of lysine diisocyanate, tetramethylxylylene diisocyanate (TMXDI), trimethylhexane diisocyanate or tetramethylhexane diisocyanate and 3 (or 4), 8 (or 9)-bis(isocyanatomethyl)-tricyclo[5.2.1.0^{2.6}]decane isomer mixtures, and also cycloaliphatic diisocyanates such as 1,4-, 1,3- or 1,2-diisocyanatocyclohexane, 4,4′- or 2,4′-di(isocyanatocyclohexyl)methane, 1-isocyanato-3,3,5-trimethyl-5-(isocyanatomethyl)cyclohexane (isophorone diisocyanate), 1,3- or 1,4-bis(isocyanatomethyl)cyclohexane, 2,4- or 2,6-diisocyanato-1-methylcyclohexane.

Preference is given to pentamethylene 1,5-diisocyanate, 1,6-diisocyanatohexane, 1-isocyanato-3,3,5-trimethyl-5-(isocyanatomethyl)cyclohexane, 4,4′-di(isocyanatocyclohexyl)methane and tolylene diisocyanate isomer mixtures. Particular preference is given to 1,6-diisocyanatohexane, 1-isocyanato-3,3,5-trimethyl-5-(isocyanatomethyl)cyclohexane and 4,4′-di(isocyanatocyclohexyl)methane.

Among the amines mentioned, amines which can be used in a gas-phase phosgenation have to meet particular requirements:

In a gas-phase phosgenation process, the reaction of amines to form the corresponding isocyanates can be carried out using amines which are such that the amine, their corresponding intermediates and the corresponding isocyanates are present in gaseous form under the selected reaction conditions. Preference is given to amines which decompose to an extent of not more than 2 mol %, particularly preferably not more than 1 mol % and very particularly preferably not more than 0.5 mol %, during the duration of the reaction under the reaction conditions. Amines, in particular diamines, based on aliphatic or cycloaliphatic hydrocarbons having from 2 to 18 carbon atoms are particularly useful here. Examples are 1,5-diaminopentane, 1,6-diaminohexane, 1-amino-3,3,5-trimethyl-5-aminomethylcyclohexane (IPDA) and 4,4′-diaminodicyclohexylmethane. Particular preference is given to using 1,6-diaminohexane (HDA).

It is likewise possible to use aromatic amines which can be converted into the gas phase without significant decomposition for the process of the invention. Examples of preferred aromatic amines are toluenediamine (TDA) as 2,4- or 2,6 isomer or mixture thereof, for example as an 80:20 to 65:35 (mol/mol) mixture, diaminobenzene, 2,6-xylidine, naphthalenediamine (NDA) and 2,4′- or 4,4′-methylenedi(phenylamine) (MDA) or isomer mixtures thereof. Among these, preference is given to the diamines and particular preference is given to 2,4- and/or 2,6-TDA.

In the gas-phase phosgenation, it is by definition desirable for the compounds occurring during the course of the reaction, i.e. starting materials (diamine and phosgene), intermediates (in particular the monocarbamyl and dicarbamyl chlorides formed as intermediates), end products (diisocyanate) and any inert compounds introduced, to remain in the gas phase under the reaction conditions. Should these or other components precipitate from the gas phase, e.g. on the reactor wall or other components of the apparatus, the heat transfer or the flow through the apparatus components affected can be altered in an undesirable fashion by these deposits. This applies particularly to amine hydrochlorides which are formed from free amino groups and hydrogen chloride (HCl), since the resulting amine hydrochlorides precipitate easily and are difficult to revaporize.

The phosgene used in the process of the invention is in principle not subject to any restrictions.

It can be preferable to use phosgene having a content of less than 50 ppm of bromine and iodine, as is disclosed in WO 01/00569.

Inerts are compounds which can be used as solvents in the liquid-phase phosgenation or as inert additions in the gas-phase phosgenation.

For the present purposes, inert means that less than 5 mol % of the solvent used is changed chemically during the course of the reaction per reaction pass, preferably less than 3 mol %, particularly preferably less than 2 mol % and very particularly preferably less than 1 mol %.

As solvents, preference is given to chlorobenzene, o- or p-dichlorobenzene, trichlorobenzene, chlorotoluenes, chloroxylenes, chloroethylbenzene, chloronaphthalenes, chlorobiphenyls, methylene chloride, perchloroethylene, toluene, xylenes, hexane, decahydronaphthalene, diethyl isophthalate (DEIP) and other carboxylic esters as are mentioned, for example, in U.S. Pat. No. 5,136,086, column 3, lines 3 to 18, tetrahydrofuran (THF), dimethylformamide (DMF), benzene and mixtures thereof. The isocyanate prepared or a stream from the process can also be used as solvent. Particular preference is given to chlorobenzene and dichlorobenzene.

The inert medium is a medium which is present in gaseous form in the reaction space at the reaction temperature and does not react with the compounds occurring during the course of the reaction. The inert medium is generally mixed with amine and/or phosgene prior to the reaction, but can also be introduced separately from the starting material streams. For example, it is possible to use nitrogen, noble gases such as helium or argon or aromatics such as chlorobenzene, chlorotoluene, o-dichlorobenzene, toluene, xylene, chloronaphthalene, decahydronaphthalene, carbon dioxide or carbon monoxide. Preference is given to nitrogen and/or chlorobenzene as inert medium.

In general, the inert medium is used in such an amount that the ratio of the gas volumes of inert medium to amine or to phosgene is from >0.0001 to 30, preferably from >0.01 to 15, particularly preferably from >0.1 to 5.

### Reaction Conditions for Gas-Phase Phosgenation

The reaction of phosgene with amine in the process of the invention takes place in the gas phase. For the present purposes, reaction in the gas phase means that the starting material streams and intermediates react with one another to form the products in the gaseous state and remain in the gas phase to an extent of at least 95%, preferably at least 98%, particularly preferably at least 99%, very particularly preferably at least 99.5%, in particular at least 99.8% and especially at least 99.9%, during the reaction while passing through the reaction space.

The starting amines are vaporized and heated to from 200° C. to 600° C., preferably from 300° C. to 500° C., before carrying out the process of the invention and are, if appropriate, diluted with an inert gas or with the vapor of an inert solvent before being passed via the mixing device into the reactor. For the present purposes, the reactor is the technical apparatus which comprises the reaction space.

The phosgene used in the phosgenation is, if appropriate, diluted with an inert gas or with the vapor of an inert solvent and likewise heated to a temperature in the range from 200° C. to 600° C., preferably from 300° C. to 500° C., before carrying out the process of the invention.

The reaction of phosgene with amine in the reaction space in the gas-phase phosgenation generally takes place at absolute pressures of from >0.1 bar to <20 bar, preferably from 0.5 bar to 15 bar and particularly preferably from 0.7 to 10 bar. In the case of the reaction of (cyclo)aliphatic amines, the absolute pressure is very particularly preferably from 0.7 bar to 5 bar, in particular from 0.8 to 3 bar and especially from 1 to 2 bar. However, the gas-phase phosgenation can also be carried out at moderate pressures of from 3 to 20 bar, as described in WO 2004/026813.

### Reaction Conditions for Liquid-Phase Phosgenation

In contrast, the reaction of amine and phosgene in the liquid-phase phosgenation generally takes place at from 20° C. to 250° C., preferably from 40 to 230° C. The pressure is generally in the range from 1.0 bar to 80 bar abs.

It is critical that the reaction of amine with phosgene takes place predominantly in the liquid phase. In addition, it is quite possible for a gas phase which comprises, for example, predominantly hydrogen chloride also to be present.

Regardless of whether the reaction is carried out as a liquid-phase or gas-phase phosgenation, the starting compounds are firstly mixed with one another in at least one mixing device and subsequently reacted in at least one reaction space. In the gas-phase phosgenation, the output from the reaction space is subsequently cooled in a “quench” by bringing it into contact with a solvent in order to stop the reaction.

The output from the reaction space in the liquid-phase phosgenation or the output from the quench in the gas-phase phosgenation is then usually purified by distillation.

### Reaction Procedure for Liquid-Phase Phosgenation

The mixing of the starting material streams, in which the amine can also be used as hydrochloride, is frequently carried out in a suitable specific mixing device which has short mixing times.

The mixing time in such mixing devices is usually from 0.0001 s to 2 s, preferably from 0.0005 to 1 s, particularly preferably from 0.001 to 0.5 s, very particularly preferably from 0.005 to 0.2 s and in particular from 0.007 to 0.1 s.

The mixing time is the time which elapses from commencement of the mixing process to the point at which 97.5% of the fluid elements of the resulting mixture have a mixing fraction which, based on the value of the theoretical end value of the mixing fraction of the resulting mixture on achieving a state of perfect mixing, deviate by less than 2.5% from this end value of the mixing fraction. (For information on the concept of the mixing fraction, see, for example, J. Warnatz, U. Maas, R. W. Dibble: Verbrennung, Springer Verlag, Berlin Heidelberg New York, 1997, 2nd Edition, p. 134.)

As mixing device, preference is given to using a mixing circuit, a stirred vessel, a mixing pump or a nozzle mixing device, for example coaxial mixing nozzles, Y- or T-mixers, or a vortex impinging jet mixing apparatus, preferably a mixing circuit, a stirred vessel, a mixing pump or a nozzle mixing device.

When a mixing circuit or a stirred vessel is used as mixing device, it is important that the amine solution is injected at a high velocity. The velocities are usually in the range from 10 to 100 m/s, preferably from 20 to 80 m/s.

Preference is given to using a mixing nozzle and a mixing pump as mixing device. Particular preference is given to using a mixing nozzle as mixing device. It is important here that both the phosgene stream and the amine stream are introduced into the mixing nozzle at high velocity. The velocities are in the range from 10 to 100 m/s, preferably from 20 to 80 m/s.

Here, the pressure in the amine feed line and phosgene feed line to the nozzle is considerably higher than in the outlet of the mixing nozzle, but usually not higher than 110 bar abs, preferably not higher than 100 bar abs, particularly preferably from 10 to 95 bar abs and very particularly preferably from 15 to 90 bar abs.

The pressure at the outlet of the mixing device is in the range from 10 to 100 bar, preferably from 15 to 80 bar, particularly preferably from 30 to 70 bar.

The temperature of the output from the mixing device is generally in the range from 25 to 240° C., preferably 30-190° C. and particularly preferably 40-160° C. The output from the mixing device can be brought to the temperature desired in the reaction space by means of a heat exchanger before being introduced into the reaction space.

In the reaction space, the reaction is completed and a major part of the carbamoyl chloride formed is dissociated to form the isocyanate.

The reaction space can comprise backmixed or unbackmixed reactors and reactive distillation columns.

Backmixed reactors are, for example, stirred vessels, cascades of stirred vessels comprising from 2 to 4 stirred vessels, loop reactors or unstirred vessels.

Largely backmixing-free reactors are, for example, tube reactors. This is achieved, for example, by the ratio of the diameter of the tube reactor to its length or by means of internals such as perforated plates, slotted plates or static mixers. The freedom from backmixing is preferably achieved by the ratio of length to diameter of the tube reactor. Suitable tube reactors are all tubes whose length to diameter ratio is greater than 5, preferably greater than 6, particularly preferably greater than 10.

A gas phase obtained during the reaction can be conveyed in cocurrent or countercurrent to the liquid phase.

Reactive distillation columns are of a construction known per se and have the usual internals. Possible column internals are in principle all customary internals, for example trays, ordered packings and/or beds of random packing elements. Preferred trays are bubble cap trays, sieve trays, valve trays, Thormann trays and/or dual-flow trays, and preferred beds of random packing elements are those comprising rings, helices, saddle bodies, Raschig rings, Intos rings or Pall rings, barren or Intalox saddles, Top-Pak etc., or braids. Preference is given to using trays, particularly preferably bubble cap trays.

Such columns usually have 10-80 theoretical plates.

The mean total residence time in the reaction space in liquid-phase phosgenation is generally from 20 min to 18 h, preferably from 30 min to 15 h, particularly preferably from 50 min to 11 h.

The conversion in the reaction space is virtually quantitative.

### Reaction Procedure for Gas-Phase Phosgenation

To carry out the reaction in the gas-phase phosgenation, the starting materials and, if appropriate, inerts are generally mixed with one another by means of at least one mixing device.

The mixing device can preferably be a static mixing device, for example a nozzle mixing device, for example coaxial mixing nozzles, Y- or T-mixers, jet mixers or mixing tubes.

In the case of a jet mixer, one component (preferably the amine) is fed at high velocity through a concentric tube having a small diameter (nozzle) into the other component (here phosgene) in a mixing tube.

Reactors can be, for example, cylindrical reaction spaces without internals and without moving parts.

An embodiment of a mixing/reaction unit is described in EP 1275639 A1, there particularly in paragraphs [0013] to [0021] and the example together with FIG. 1, which is hereby incorporated by reference into the present disclosure. However, in contrast to the disclosure there, preference is given to introducing the amine through the inner tube and the phosgene as outer stream.

An embodiment of a mixing/reaction unit is described in EP 1275640 A1, there particularly in paragraphs [0010] to [0018] and the example together with FIG. 1, which is hereby incorporated by reference into the present disclosure. However, in contrast to the disclosure there, preference is given to introducing the amine through the inner tube and the phosgene as outer stream.

A further embodiment of a mixing/reaction unit is described in EP 1319655 A2, there particularly in paragraphs [0015] to [0018] and the example together with FIG. 1, which is hereby incorporated by reference into the present disclosure.

It can be useful to install flow equalizers as described in EP 1362847 A2, there particularly in paragraphs [0008] to [0026] and the example together with FIG. 1, which is hereby incorporated by reference into the present disclosure.

The use of a plurality of parallel nozzles as described in EP 1449826 A1, there particularly in paragraphs [0011] to [0027] and Example 2 together with FIGS. 1 to 3, which is hereby incorporated by reference into the present disclosure, is also conceivable.

It can be useful to increase the degree of turbulence for mixing by means of internals which induce a rotational motion, as described in EP 1526129 A1, there particularly in paragraphs [0008] to [0026] and the example together with FIGS. 1 to 3, which is hereby incorporated by reference into the present disclosure.

A further embodiment of a mixing/reaction unit is described in DE 10359627 A1, there particularly in paragraphs [0007] to [0025] and Example 1 together with the figure, which is hereby incorporated by reference into the present disclosure.

A preferred embodiment of a mixing nozzle is a slit mixing nozzle as described in the European patent application number 06123631.1 filed on Nov. 7, 2006, there particularly from page 3, line 28 to page 15, line 35, and a reaction space as is described there from page 15, line 39 to page 23, line 38, together with the figures, which is hereby incorporated by reference into the present disclosure.

A particularly preferred embodiment of a mixing nozzle is an annular gap mixing nozzle as is described in the international patent application WO 2007/028715, there particularly from page 2, line 23 to page 11, line 22, and a reaction space as described there from page 11, line 26 to page 21, line 15, together with FIG. 2, which is hereby incorporated by reference into the present disclosure.

The quench can, for example, be configured as described in EP 1403248 A1, there particularly in paragraphs [0006] to [0019] and the example together with FIGS. 1 and 2, which is hereby incorporated by reference into the present disclosure.

The quench can, for example, be configured as described in the European patent application number 06123629.5 filed on Nov. 7, 2006, there particularly from page 3, line 30 to page 11, line 37 together with Example 1 and the figures, which is hereby incorporated by reference into the present disclosure.

The quench can, for example, be configured as described in the European patent application number 06123621.2 filed on Nov. 7, 2006, there particularly from page 3, line 26 to page 16, line 36 together with Example 1 and the figures, which is hereby incorporated by reference into the present disclosure.

The quench can preferably be configured as described in WO 2005/123665, there particularly from page 3, line 10 to page 8, line 2 and the example, which is hereby incorporated by reference into the present disclosure.

It is important for the purposes of the invention for surfaces of the apparatus in which the reaction takes place which come into contact with fluid to have a mean peak-to-valley height Rz in accordance with DIN EN ISO 4287 (October 1998 version) of not more than 10 μm, preferably not more than 8 μm, particularly preferably not more than 6 μm, very particularly preferably not more than 4 μm, in particular not more than 2 μm and especially not more than 1 μm.

Lower deposit formation is observed on such surfaces which according to the invention have a low roughness than on rougher surfaces.

In particular, surfaces which come into contact with the two starting materials phosgene and amine and/or come into contact with one of the intermediates in the reaction, for example the corresponding carbamoyl chlorides, preferably surfaces which come into contact with the two starting materials phosgene and amine, should have a low roughness according to the invention.

It can also be useful for surfaces which come into contact with only one starting material, i.e. either amine or phosgene, for example in feed lines, to be given a low roughness according to the invention.

It can also be useful for surfaces which come into contact with essentially pure product, i.e. the isocyanate, to be given a low roughness according to the invention.

In a preferred embodiment, at least the surfaces of the mixing device which come into contact with fluid are given a low roughness according to the invention.

In a particularly preferred embodiment, at least the surfaces of the reaction space which come into contact with fluid are given a low roughness according to the invention.

It can also be useful for the quench additionally to be given a low roughness according to the invention.

The term “fluid” comprises both a liquid phase and a gas phase.

For the purposes of making a distinction, the mixing space can be considered to be the region of the reaction space in which mixing of the starting materials takes place to an extent of 99%. In a preferred embodiment of the present invention, the conversion in the mixing space, i.e. the consumption of the amine used, is less than 15%. Here, the degree of mixing is reported as ratio of the difference between the locally averaged mixing fraction and the initial mixing fraction before mixing to the difference between the mean final mixing fraction after mixing and the initial mixing fraction before mixing. For information on the concept on the mixing fraction, see, for example, J. Warnatz, U. Maas, R. W. Dibble: Verbrennung, Springer Verlag, Berlin Heidelberg New York, 1997, 2nd edition, p. 134.

The reaction space is the volume in which at least 98% of the conversion, i.e. the consumption of the amine used, takes place, preferably at least 99%, particularly preferably 99.5%, very particularly preferably 99.7%, in particular 99.9% and especially 99.99%.

Apart from the averaged peak-to-valley heights Rz indicated, it can be useful to adhere to further parameters, for example the arithmetic mean roughness values Ra in accordance with DIN EN ISO 4287 (October 1998 version), which should generally be not more than 1.5 μm, preferably not more than 1.0 μm and particularly preferably not more than 0.8 μm.

The values of Rz and, if appropriate, Ra are preferably measured using electric contact stylus instruments. This is preferably carried out in accordance with DIN EN ISO 4288.

Here, an electric contact stylus instrument having a highly sensitive diamond stylus traces the profile along the standardized tracing stretch. A reference line, known as the mean line, is automatically averaged. Basically, a two-dimensional recording of the approximate image of the workpiece surface is recorded by the instrument. The highly sensitive stylus travels over the surface contour during the roughness measurement.

Further measurement methods are known in principle and may be found in the technical literature (for example Dubbel “Taschenbuch für den Maschinenbau”, Springer-Verlag).

Suitable materials for contact with the reaction mixture are, for example, metals such as steel, in particular alloy steel, tantalum, nickel, nickel alloys, silver or copper, glass, ceramic, enamels or homogeneous or heterogeneous mixtures and components made thereof. Preference is given to using steel apparatuses, particularly preferably steel reactors.

The apparatuses are preferably made at least partly of corrosion-resistant materials at the thermally stressed places at which the apparatus is exposed to the reaction mixture. Such materials include, in particular, stainless steels and wrought nickel alloys.

DIN-EN 10088-1 in the version of August 1995 defines stainless steels as steels comprising at least 10.5% of chromium and not more than 1.2% of carbon. According to the invention, preference is then given to apparatuses which are made at least partly of austenitic and/or austenitic-ferritic steels.

Austenitic steels are steels having an austenitic lattice type (γ phase) at 20° C. They preferably have a Cr content of from 16 to 28% and an Ni content of from 3.5 to 32% and also, if appropriate, proportions of S (up to 0.35%), P (up to 0.045%), Mo (up to 7%), Si (up to 4.5%), Cu (up to 4%), N (up to 0.25%) and/or Mn (up to 10.5%), and also possibly Ti (up to 0.7%) and/or Nb (up to 1%). The carbon content is generally less than 0.15%. Among these, the high alloy austenitic 18/8 chromium-nickel steels are particularly preferred.

Austenitic-ferritic steels have a two-phase microstructure composed of ferrite and austenite and have a proportion of ferrite of about 60%. The Cr content is usually 19-28%, the Ni content is 3.5-8%, the Mo content is up to 4.5% and the steel may, if appropriate, comprise proportions of Mn (up to 2%), Cu (up to 2.5%), N (up to 0.35%), W (up to 1%), S (up to 0.015%), Si (up to 1%) and/or P (up to 0.035%). The carbon content is generally less than 0.05%.

Very particularly preferred materials are the austenitic and austenitic-ferritic materials described in DIN-EN 10088-1, in particular the materials 1.4539 (Falk steel), 1.4541, 1.4571 and 1.4462, and also Hastelloy A and C and zirconium. The abovementioned materials in accordance with DIN-EN 10088 correspond approximately to the following materials in accordance with AISI (American Iron and Steel Institute), UNS (Unified Numbering System), SS (Swedish Standard), AFNOR (Association Francaise de Normalisation), BS (British Standard) and JIS (Japanese Industrial Standards):

### 1.4462 (X 2 CrNiMoN 22 5 3): UNS: S 31803, SS: 2377, AFNOR: Z 5 CNDU 21.08, JIS: SUS 329 J3L

### 1.4539 (X 1 NiCrMoCuN 25 20 5): UNS: N 08904, SS: 2562, AFNOR: Z 1 NCDU 25.20 1.4541 (X 6 CrNiTi 18 10): AISI: 321, UNS: S 32100, SS: 2337, AFNOR: Z 6 CNT 18.10, BS: 321 S 31, JIS: SUS 321

### 1.4571 (X 6 CrNiMoTi 17 12 2): AISI: 316 Ti, UNS: S 31635, SS: 2350, AFNOR: Z 6 CNDT 17.12, BS: 320 S 31, JIS: SUS 316 Ti

Among the materials listed, those having relatively high chromium, copper, molybdenum and/or nickel contents are advantageous.

Apart from the abovementioned stainless steels, it is likewise possible to use nickel and wrought nickel alloys (material designations 2.4xxx in accordance with DIN 17744:2002-09). Preference is given to utilizing heat- and corrosion-resistant alloys having a molybdenum content of from 6% by weight to 25% by weight and a chromium content of from >6% by weight to 26% by weight. Apart from small amounts of other metals, these materials can comprise relatively large proportions of cobalt, copper, iron, manganese, niobium and/or tantalum and also tungsten.

Furthermore, it is possible to use copper-comprising wrought nickel alloys having the material designations 2.4xxx in accordance with DIN 17744:2002-09. These comprise nickel together with, in particular, copper; apart from small amounts of other metals, these materials can comprise relatively high proportions of iron.

Particular preference is given to the following materials: 2.4602 (NiCr21 Mo14W), 2.4606 (NiCr21 Mo16W), 2.4610 (NiMo16Cr16Ti), 2.4619 (NiCr22Mo7Cu), 2.4819 (NiMo16Cr15W), 2.4856 (NiCr22Mo9Nb), 2.4360 (NiCu30Fe) and 2.4361 (LCNiCu30Fe).

These materials are frequently marketed under the trade names HASTELLOY®, INCONEL®, MONEL® and others.

The apparatuses having the low roughnesses according to the invention are generally produced by means of multistage manufacture using parting manufacturing methods. Thus, the first manufacturing step such as drilling, milling or turning can be followed by further manufacturing steps. These can likewise be drilling, milling or turning, but then with altered parameters, e.g. a reduced advance rate (fine finishing). Other parting manufacturing methods such as grinding, mechanical polishing and/or electropolishing, honing or lapping can likewise be employed. However, an alternative is to smooth rough surfaces by material-application methods such as coating, plating, galvanizing or the like.

The formation of deposits in liquid-phase phosgenation and gas-phase phosgenation, preferably gas-phase phosgenation, can be reduced by means of apparatuses having a low roughness according to the invention.

The invention is illustrated by the following examples:

### EXAMPLE 1

### Comparison

In an experimental plant for the phosgenation of an isomer mixture of toluenediamine (ratio of 2,4 isomer to 2,6 isomer about 80:20) to form tolylene diisocyanate in the liquid phase, a mixture of toluenediamine and monochlorobenzene was fed in via a central capillary having a diameter of 0.6 mm. A molar excess of phosgene relative to the amine is conveyed in an annular gap around this capillary. The channels open into a reaction vessel in such a way that the openings are immersed in the liquid reaction mixture.

During operation of the plant, blockage of the capillary opening with solids occurred within a few hours. The mean roughness value Ra for the nozzle (measured on the outside of the inner capillary in accordance with DIN EN ISO 4288, 4287, 3274) was 1.97 μm.

### EXAMPLE 2

### According to the Invention

After coating of the capillary of Example 1 with a glass coating having a mean roughness Ra of about 0.54 μm stable operation for four weeks without blockages could be achieved.

1. A process for preparing a diisocyanate by the reaction of the corresponding amine with phosgene, optionally in the presence of inerts, wherein the reaction takes place at least partly in an apparatus whose fluid contact surface has an average peak-to-valley height Rz in accordance with DIN EN ISO 4287 (October 1998 version) of not more than 10 μm.

2. The process according to claim 1, wherein the isocyanate is selected from the group consisting of pentamethylene 1,5-diisocyanate, 1,6-diisocyanatohexane, 1-isocyanato-3,3,5-trimethyl-5-(isocyanatomethyl)cyclohexane, 4,4′-di(isocyanatocyclohexyl)methane and tolylene diisocyanate isomer mixtures.

3. The process according to claim 1, wherein, in the reaction, the starting material streams and intermediates react with one another to form the products in the gaseous state and remain in the gas phase to an extent of at least 95% during the reaction while passing through the reaction space.

4. The process according to claim 1, wherein the reaction of amine and phosgene occurs at from 20° C. to 250° C. and a pressure of from 1.0 bar to 80 bar abs.

5. The process according to claim 1, wherein the average peak-to-valley height Rz in accordance with DIN EN ISO 4287 (October 1998 version) is not more than 4 μm.

6. The process according to claim 1, wherein the arithmetic mean roughness value Ra in accordance with DIN EN ISO 4287 (October 1998 version) is not more than 1.5 μm.

7. The process according to claim 1, wherein the fluid contact surface comprises a material selected from the group consisting of steel, alloy steel, tantalum, nickel, nickel alloy, silver, copper, glass, ceramic, enamel and homogeneous or heterogeneous mixtures and components made thereof.

8. The process according to claim 1, wherein the apparatus having a surface of low roughness is at least the reactor.

9. A mixing device for use in liquid-phase phosgenation or gas phase phosgenation having a fluid contact surface which has an average peak-to-valley height Rz in accordance with DIN EN ISO 4287 (October 1998 version) of not more than 10 μm.

10. A reactor for use in liquid-phase phosgenation or gas-phase phosgenation having a fluid contact surface which has an average peak-to-valley height Rz in accordance with DIN EN ISO 4287 (October 1998 version) of not more than 10 μm.

11. A quench apparatus for use in liquid-phase phosgenation or gas phase phosgenation having a fluid contact surface which has an average peak-to-valley height Rz in accordance with DIN EN ISO 4287 (October 1998 version) of not more than 10 μm.
